(19) United States
(12) Patent Application Publication
(10) Pub. No.: US 2004/0059355 A1
(43) Pub. Date: Mar. 25, 2004

(54) Clamping device

(76) Inventor: Wen-Hwa Kuo, Taipei (TW)

Correspondence Address: VENABLE, BAETJER, HOWARD AND CIVILETTI, LLP, P.O. BOX 34385, WASHINGTON, DC, 20043-9998, US

(21) Appl. No.: 10/253,585

(22) Filed: Sep. 25, 2002

(51) Int. Cl. A61B 17/08

(52) U.S. Cl. 606/151

(57) ABSTRACT

A clamping device includes a body having a centrally defined passage and two holes formed on opposite sides of the body to communicate with the passage, a handle rotatably received in the passage and having an abutting member extending into an outer periphery of the handle, a clamping member having two distal ends extending into the passage and out of the two holes respectively to be abutted by the abutting member, and a ratchet member provided to prevent undesired rotation between the body and the handle.

### BACKGROUND OF THE INVENTION

1. Field of the Invention

The present invention relates to a clamping device, and more particularly to a medical use clamping device, with which parts for medical use are combined together securely.

2. Description of Related Art

A conventional manner of combining parts together depends on users making knots to tie the parts together. When making these knots, often the user has troubles trying to fasten the parts by using a rope or the like. It is because the conventional manner depends entirely on the user's manual force and because all ropes are elastic so that when the rope or the like is fastened to a certain degree, the elasticity of the rope will somehow hinder the tightness of the parts.

To overcome the shortcomings, the present invention tends to provide a clamping device to mitigate and obviate the aforementioned problems.

### SUMMARY OF THE INVENTION

The primary objective of the present invention is to provide a clamping device to tie things together.

Another objective of the present invention is to provide a ratchet member on the clamping device to overcome elasticity of the clamping member.

In order to accomplish the foregoing objective, the clamping device of the present invention includes a body, a handle rotatably received in the body, a clamping member forming a loop on top of the body and having two distal ends extending into the body and an abutting member extending into an outer periphery of the body to abut the two distal ends. Rotation of the handle gradually reduces the size of the loop so that parts inside the loop will be securely tied together. Furthermore, a ratchet device is provided on a side of the body so that there is no relative rotation of the body to the handle.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view showing parts of the clamping device of the present invention;

FIG. 2 is a side plan view showing the assembled clamping device in FIG. 1;

FIG. 3 is a schematic enlarged view showing that the abutting member is abutting the two distal ends of the clamping member;

FIG. 4 is a schematic view showing the ratchet member used in the clamping device of the present invention;

FIG. 5 is a schematic view showing that after the abutting member is abutting the two distal ends of the clamping member, rotation of the handle will reduce the size of the loop;

FIG. 6A is a schematic side plan view showing another preferred embodiment of the clamping device of the present invention; and

FIG. 6B is an enlarged side plan view showing the head of the body of the embodiment in FIG. 6A.

### DETAILED DESCRIPTION OF THE PREFERRED EMBODIMENT

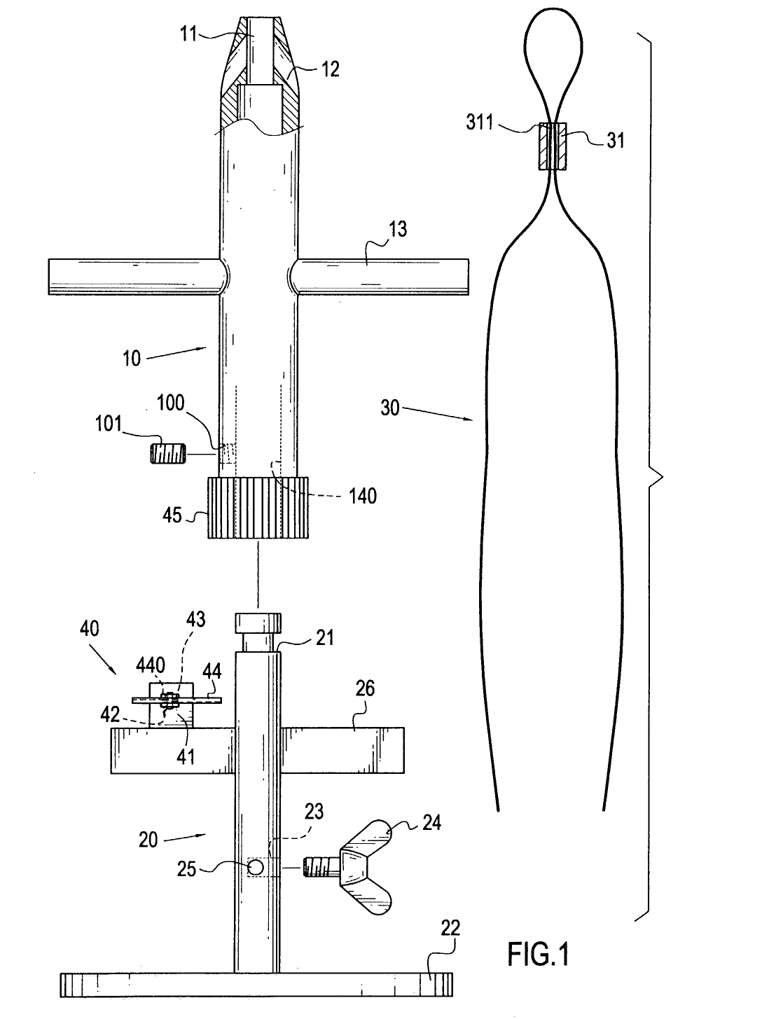

With reference to FIG. 1, the clamping device in accordance with the present invention includes a body (10), a handle (20) and a clamping member (30). A ratchet member (40) is provided on a side of the body (10).

The body (10) has a centrally defined passage (11) in a head of the body (10), two holes (12) oppositely defined on a side face of the head of the body (10) to respectively communicate with the passage (11). A pair of arms (13) respectively and oppositely extends outward from the body (10). It is noted that an orifice (100) is defined through a side face of the body (10) to allow an extension of a stop (101).

The handle (20) has a first end with an annular groove (21) defined around an outer periphery of the first end and a second end with a holding portion (22) extending out to opposite directions. A first aperture (23) is defined in a side face of the handle (20) to allow an extension of an abutting member (24). A second aperture (25) is orthogonally defined relative to the first aperture (23). A seat (26) integrally extend out from the handle (20) to opposite directions.

The clamping member (30) is a rope having two distal ends and a retainer (31) having a path (311) defined through the retainer (31) to allow the two distal ends to extend through the path (311).

The ratchet member (40) includes two supports (41) horizontally seated on a top face of the seat (26), a pin (42) extending through the two supports (41), a spring (43) and a finger (44) pivotally connected between the two supports (41). The finger (44) has a pin hole (440) defined to correspond to the pin (42). A ratchet gear (45) is formed around the outer periphery of the body (10) to correspond to the finger (44).

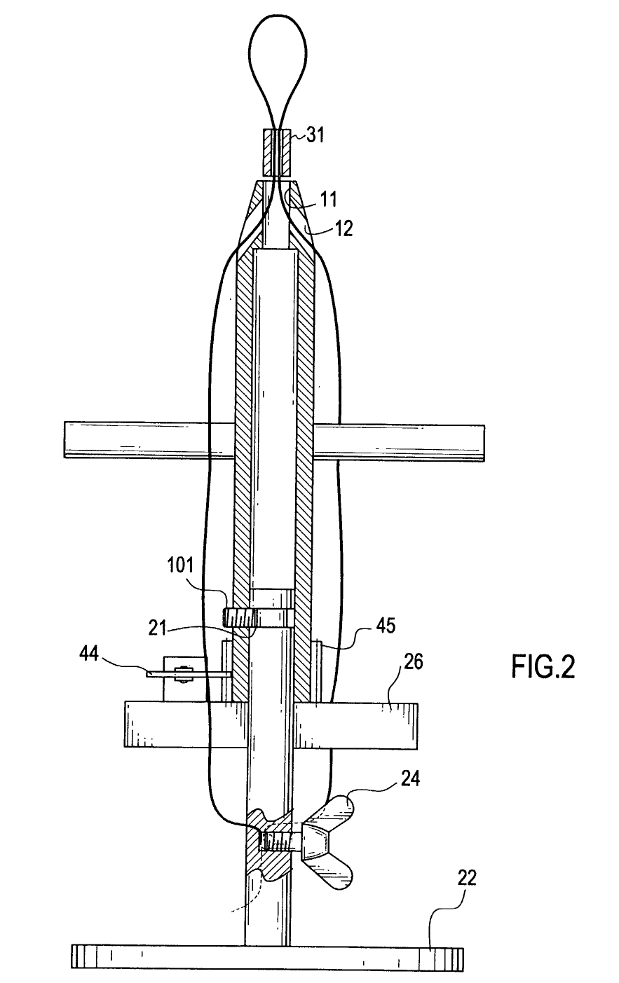

With reference to FIG. 2, when the clamping device of the present invention is in assembly, the handle (20) extends into the passage (11). The stop (101) then extends through the orifice (100) and into the annular groove (21) of the handle (20) to prevent the handle (20) from detachment with the body (10). The spring (43) is mounted around the pin (42). The pin (42) extends through the pin hole (440) of the finger (44). A first end of the spring (43) abuts the top face of the seat (26) and a second end of the spring (43) abuts a bottom face of the finger (44) to provide a recoil force to the finger (44). Then the two distal ends of the clamping member (30) both extend into the passage (11) and respectively extend out of the body (10) from a corresponding one of the holes (12). Therefore the two distal ends of the clamping member (30) extend into the second aperture (25) which is orthogonal to the first aperture (23).

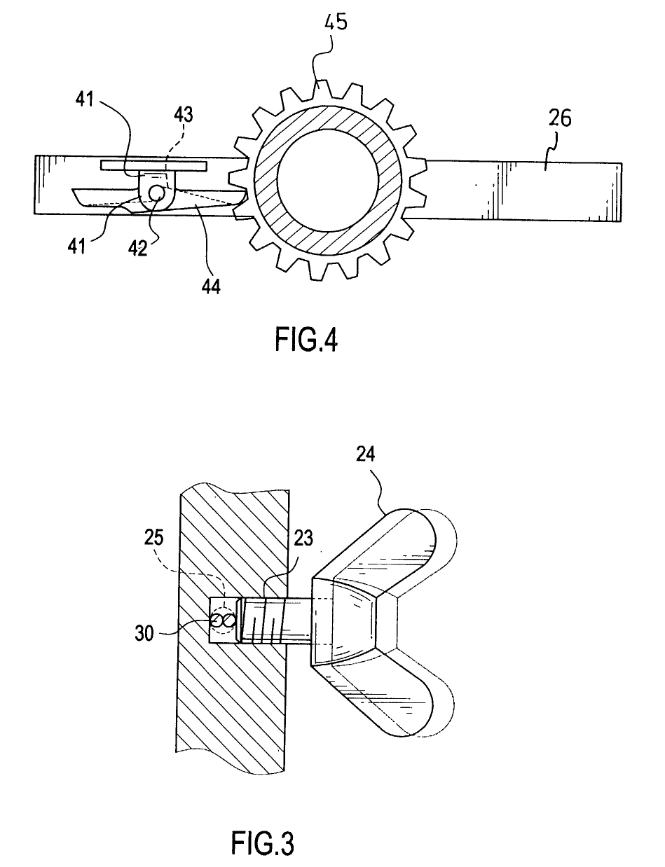

With reference to FIG. 3, it is to be noted that the first aperture (23) has a diameter larger than a diameter of the second aperture (25) so that after the two distal ends of the clamping member (30) extend into the second aperture (25) and the abutting member (24) extends into the first aperture (23), the two distal ends of the clamping member (30) are securely abutted by a distal end of the abutting member (24) against an inner face defining the first aperture (23) of the handle (20). In order to have a better clamping effect, the two distal ends of the clamping member (30) preferably extend to the second aperture (25) from different sides of the arm (13).

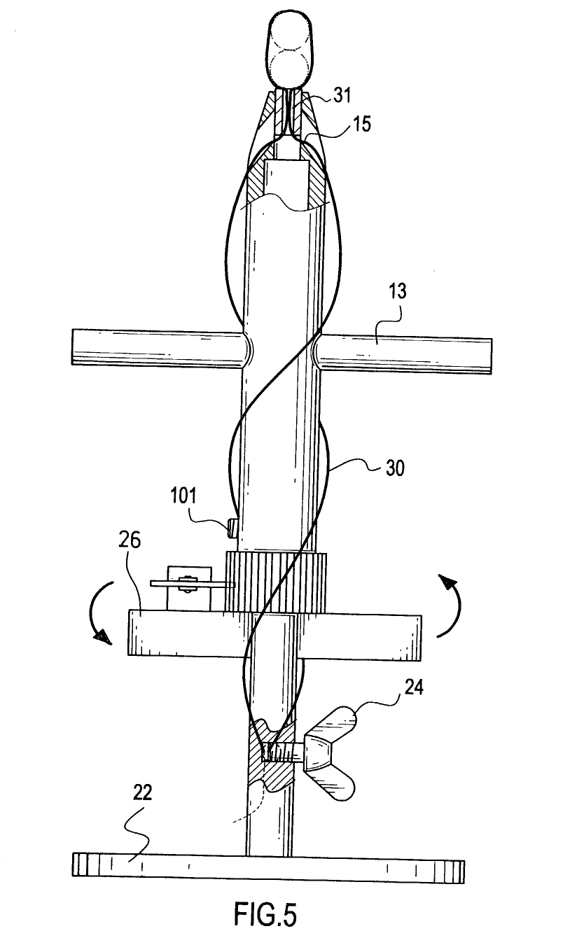

With reference to FIGS. 4 and 5, after the assembly of the clamping device of the present invention, the user rotates the handle (20) relative to the body (10). The clamping member (20) is then around the outer periphery of the body (10) and of the handle (20). As the handle (20) rotates, a distal end of the finger (44) abuts the ratchet gear (45) to avoid any reverse rotation between the body (10) and the handle (20). Furthermore, the head of the body (10) has a shoulder (15) formed inside the passage (11) so that when the retainer (31) is received in the passage (11), the retainer (31) is stopped by the shoulder (15). Therefore, when the rotation of the handle (20) continues, the loop of the clamping member (30) above the retainer (31) reduces and thus objects (in dashed lines) enclosed by the clamping member (30) are securely combined by the clamping member (30). After the objects are securely combined by the clamping member (30), the user is then able to cut off the clamping member (30) right beneath the retainer (31).

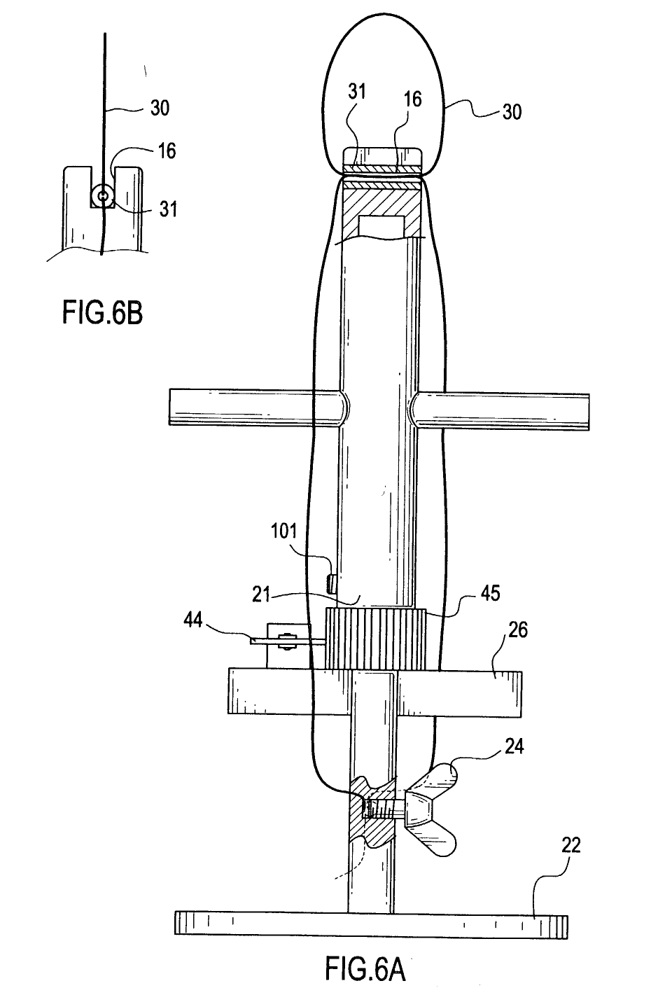

With reference to FIGS. 6A and 6B, another preferred embodiment of the clamping device is shown, wherein the head of the body (10) has a laterally defined through hole (16) defined to allow the extension of the two distal ends of the clamping member (30).

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A clamping device comprising:
a body having a centrally defined passage and two holes formed on opposite sides of the body to communicate with the passage;
a handle rotatably received in the passage and having an abutting member extending into an outer periphery of the handle;
a clamping member having two distal ends extending into the passage and out of the two holes respectively to be abutted by the abutting member; and
a ratchet member provided to prevent undesired rotation between the body and the handle.

2. The clamping device as claimed in claim 1, wherein the body has an orifice defined in an outer periphery of the body to receive therein a stop, the handle has an annular groove defined around a distal end of the handle to correspond to the stop, whereby after the stop is received in the corresponding annular groove, the handle is securely connected to the body.

3. The clamping device as claimed in claim 1, wherein the handle has a first aperture defined through an outer periphery of the handle to receive an extension of the abutting member and a second aperture orthogonally defined relative to the first aperture to receive therein the two distal ends of the clamping member so that the abutting member is able to abut the two distal ends of the clamping member against an inner face defining the first aperture.

4. The clamping device as claimed in claim 2, wherein the handle has a first aperture defined through an outer periphery of the handle to receive an extension of the abutting member and a second aperture orthogonally defined relative to the first aperture to receive therein the two distal ends of the clamping member so that the abutting member is able to abut the two distal ends of the clamping member against an inner face defining the first aperture.

5. The clamping device as claimed in claim 1, wherein the clamping member has a retainer received in the passage and provided with a path defined through the retainer to allow the two distal ends of the clamping member to extend through the path, the body has a shoulder formed in the passage to stop the retainer so that a loop formed by the clamping member above the retainer is reduced when the handle is rotated relative to the body to wind the clamping member around the body and the handle.

6. The clamping device as claimed in claim 2, wherein the clamping member has a retainer received in the passage and provided with a path defined through the retainer to allow the two distal ends of the clamping member to extend through the path, the body has a shoulder formed in the passage to stop the retainer so that a loop formed by the clamping member above the retainer is reduced when the handle is rotated relative to the body to wind the clamping member around the body and the handle.

7. The clamping device as claimed in claim 3, wherein the clamping member has a retainer received in the passage and provided with a path defined through the retainer to allow the two distal ends of the clamping member to extend through the path, the body has a shoulder formed in the passage to stop the retainer so that a loop formed by the clamping member above the retainer is reduced when the handle is rotated relative to the body to wind the clamping member around the body and the handle.

8. The clamping device as claimed in claim 1, wherein the ratchet member includes a ratchet gear formed on a bottom end of the body and a finger pivotally formed on the handle and having a distal end abutted to the ratchet gear to prevent undesired rotation between the body and the handle.

9. The clamping device as claimed in claim 3, wherein the ratchet member includes a ratchet gear formed on a bottom end of the body and a finger pivotally formed on the handle and having a distal end abutted to the ratchet gear to prevent undesired rotation between the body and the handle.

10. The clamping device as claimed in claim 5, wherein the ratchet member includes a ratchet gear formed on a bottom end of the body and a finger pivotally formed on the handle and having a distal end abutted to the ratchet gear to prevent undesired rotation between the body and the handle.

11. The clamping device as claimed in claim 8, wherein the ratchet member further has a pair of supports horizontally formed on a seat that is integrally formed on the handle and extend in opposite directions, a pin extending through the pair of supports and the finger, a spring mounted around the pin, wherein a first distal end of the spring abuts a top face of the seat and a second end of the spring abuts a bottom face of the finger to provide a recoil force to the finger.

12. The clamping device as claimed in claim 9, wherein the ratchet member further has a pair of supports horizontally formed on a seat that is integrally formed on the handle and extend in opposite directions, a pin extending through the pair of supports and the finger, a spring mounted around the pin, wherein a first distal end of the spring abuts a top face of the seat and a second end of the spring abuts a bottom face of the finger to provide a recoil force to the finger.

13. The clamping device as claimed in claim 10, wherein the ratchet member further has a pair of supports horizontally formed on a seat that is integrally formed on the handle and extend in opposite directions, a pin extending through the pair of supports and the finger, a spring mounted around the pin, wherein a first distal end of the spring abuts a top face of the seat and a second end of the spring abuts a bottom face of the finger to provide a recoil force to the finger.

14. A clamping device comprising:
a body having a laterally defined through hole;
a handle rotatably received in the passage and having an abutting member extending into an outer periphery of the handle;
a clamping member having two distal ends extending out of the through hole to be abutted by the abutting member; and
a ratchet member provided to prevent undesired rotation between the body and the handle.

15. The clamping device as claimed in claim 14, wherein the body has an orifice defined in an outer periphery of the body to receive therein a stop, the handle has an annular groove defined around a distal end of the handle to correspond to the stop, whereby after the stop is received in the corresponding annular groove, the handle is securely connected to the body.

16. The clamping device as claimed in claim 14, wherein the handle has a first aperture defined through an outer periphery of the handle to receive an extension of the abutting member and a second aperture orthogonally defined relative to the first aperture to receive therein the two distal ends of the clamping member so that the abutting member is able to abut the two distal ends of the clamping member against an inner face defining the first aperture.

17. The clamping device as claimed in claim 14, wherein the clamping member has a retainer received in the through hole and provided with a path defined through the retainer to allow the two distal ends of the clamping member to extend through the path so that a loop formed by the clamping member above the retainer is reduced when the handle is rotated relative to the body to wind the clamping member around the body and the handle.

18. The clamping device as claimed in claim 14, wherein the ratchet member includes a ratchet gear formed on a bottom end of the body and a finger pivotally formed on the handle and having a distal end abutted to the ratchet gear to prevent undesired rotation between the body and the handle.

19. The clamping device as claimed in claim 18, wherein the ratchet member further has a pair of supports horizontally formed on a seat that is integrally formed on the handle and extend in opposite directions, a pin extending through the pair of supports and the finger, a spring mounted around the pin, wherein a first distal end of the spring abuts a top face of the seat and a second end of the spring abuts a bottom face of the finger to provide a recoil force to the finger.
